(19) United States
(12) Patent Application Publication
(10) Pub. No.: US 2012/0149660 A1
(43) Pub. Date: Jun. 14, 2012

(54) NOVEL N-SUBSTITUTED 5-HYDROXYPYROLLINDES AS INHIBITORS OF MDM2-P53 INTERACTIONS

(76) Inventors: Jin-Jun Liu, Warren Township, NJ (US); Tina Morgan Ross, Royersford, PA (US)

(21) Appl. No.: 13/315,335

(22) Filed: Dec. 9, 2011

Related U.S. Application Data
(60) Provisional application No. 61/421,267, filed on Dec. 9, 2010.

(51) Int. Cl. A61K 31/40; C07F 7/10; C07D 405/06; C07D 403/06; A61P 35/00; A61K 31/4025; C07F 5/02; A61K 31/4178; A61K 31/69; C07D 207/16; A61K 31/695

(52) U.S. Cl. 514/63; 548/566; 548/406; 548/517; 548/314.7; 514/428; 514/422; 548/405; 514/397; 514/64

(57) ABSTRACT

There are provided compounds of formula I

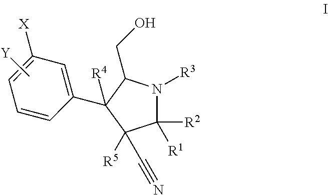

or a pharmaceutically acceptable salt thereof, wherein X, Y, R¹, R², R³, R⁴, R⁵ are as defined herein. The compounds exhibit activity as anticancer agents.

### PRIORITY TO RELATED APPLICATION(S)

This application claims the benefit of U.S. Provisional Application No. 61/421,267, filed Dec. 9, 2010, which is hereby incorporated by reference in its entirety.

### FIELD OF THE INVENTION

The present invention relates to N-Substituted hydroxypyrrolidines which act as inhibitors of MDM2-p53 interactions and are useful in the amelioration or treatment of cancer, especially solid tumors.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might also have effects in p53 mutant cells.

### SUMMARY OF THE INVENTION

One aspect of the invention is a compound of formula I

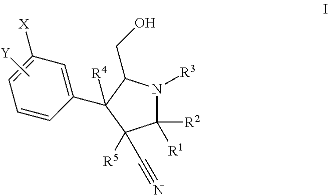

or a pharmaceutically acceptable salt thereof, wherein X, Y, R¹, R², R³, R⁴, R⁵ are as defined below.

The present invention also relates to pharmaceutical compositions comprising one or more compounds of the invention, or a pharmaceutically acceptable salt, and a pharmaceutically acceptable carrier or excipient.

The present invention further relates to a method of treating, ameliorating or preventing cancer in a mammal, preferably a human, comprising administering to said mammal a therapeutically effective amount of a compound according to the invention or a pharmaceutically acceptable salt thereof.

### DETAILED DESCRIPTION OF THE INVENTION

### Definitions

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkoxy, alkoxyl or lower alkoxy” refers to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Amino means the group —NH₂.

“Aryl” means a monovalent, monocyclic or bicyclic, aromatic carboxylic hydrocarbon radical, preferably a 6-10 member aromatic ring system. Preferred aryl groups include, but are not limited to, phenyl, naphthyl, tolyl, and xylyl.

Carboxyl or carboxy means the monovalent group —COOH. Carboxy lower alkyl means —COOR, wherein R is lower alkyl. Carboxy lower alkoxy means —COOROH wherein the R is lower alkyl.

Carbonyl means the group

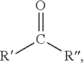

where R′ and R″ independently can be any of a number of chemical groups including alkyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “halogen” as used herein means fluorine, chlorine, bromine, or iodine, preferably fluorine and chlorine.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl.

In the case of aryl or heteroaryl which are bicyclic it should be understood that one ring may be aryl while the other is heteroaryl and both being substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

Hydroxy or hydroxyl is a prefix indicating the presence of a monovalent —O—H group.

“IC50” refers to the concentration of a particular compound required to inhibit 50% of a specific measured activity. IC₅₀ can be measured, inter alia, as is described subsequently in Example #.

“Lower” as in “lower alkenyl” means a group having 1 to 6 carbon atoms.

“Nitro” means —NO₂.

Oxo means the group ═O.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle.

In one embodiment, the present invention relates to compounds of formula I

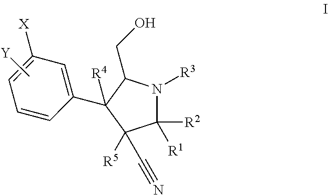

wherein
Y is selected from the group consisting of F, Cl, Br, I, cyano, nitro, lower alkyl, lower alkynyl and lower alkoxy;

### X is H, F, Cl or CF₃;

R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl, lower cycloalkyl,
R⁵ are independently selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
and enantiomers thereof or a pharmaceutically acceptable salt or ester thereof.

Preferred are compounds of formula II

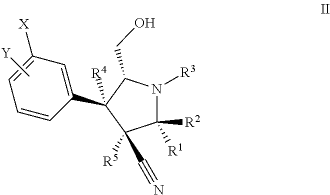

wherein
Y is selected from the group consisting of F, Cl, Br, I, cyano, nitro, lower alkyl, lower aklynyl and lower alkoxy;

### X is H, F, Cl or CF₃;

R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl, lower cycloalkyl,
R⁵ is selected from the group consisting of H, lower alkyl, substituted lower alkyl, lower cycloalkyl, substituted lower cycloalkyl, lower alkenyl, substituted lower alkenyl, lower cycloalkenyl and substituted lower cycloalkenyl
and the enantiomers thereof or a pharmaceutically acceptable salt or ester thereof.

More preferred are compounds of formula II in which R⁵ is selected from the group consisting of a substituted phenyl as shown in formula IIa:

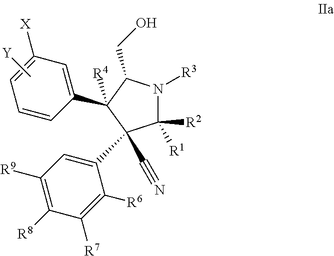

wherein,
Y is selected from the group consisting of F, Cl, Br, I, cyano, nitro, lower alkyl, lower aklynyl and lower alkoxy;

### X is H, F, Cl or CF₃;

R⁸ is selected from the group consisting of F, Cl and Br;
R⁶, R⁷ and R⁹ are H or F with the proviso that at least two of R⁶, R⁷ and R⁹ are hydrogen;
R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl, lower cycloalkyl,
and the enantiomers thereof or a pharmaceutically acceptable salt or ester thereof.

bn Further preferred are compounds of formula III in which R¹ is hydrogen, R² is selected from a group consisted of substituted lower alkyl shown as in formula III:

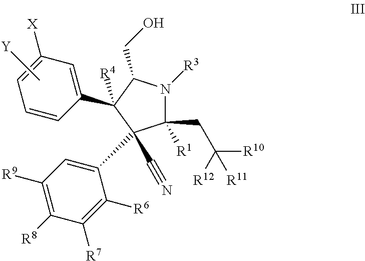

### Wherein,

Y is selected from the group consisting of F, Cl, Br, I, cyano, nitro, lower alkyl, lower aklynyl and lower alkoxy;

### X is H, F, Cl or CF₃;

R⁸ is selected from the group consisting of F, Cl and Br;
R⁶, R⁷, R⁹ are selected from H or F with the proviso that at least two of R⁶, R⁷ and R⁹ are hydrogen;
R¹⁰, R¹¹ are both methyl, or linked to form a cyclopropyl, cyclobutyl or cyclopentyl group; R¹² is (CH₂)ₘ—R¹³;
m is selected from 0, 1 or 2;
R¹³ is selected from hydrogen, hydroxyl, lower alkyl, lower alkoxy, lower cycloalkenyl, substituted cycloalkenyl, lower cycloalkyl, substituted alkylhydroxyalkylamino, substituted cycloalkyl, aryl, substituted aryl, hetereoaryl, substituted heteroaryl, hetereocycle or substituted heterocycle;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
and enantiomers thereof or a pharmaceutically acceptable salt or ester thereof.

Further preferred are compounds of formula II in which R⁵ is an heteroaryl as shown in formula IV,

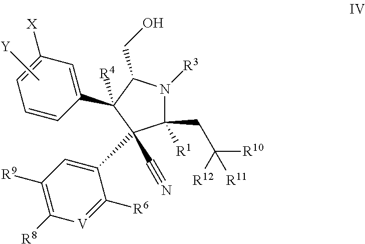

wherein,
Y is selected from the group consisting of F, Cl, Br, I, cyano, nitro, lower alkyl, lower aklynyl and lower alkoxy;

### X is H, F, Cl or CF₃;

### V is N

R⁸ is selected from the group consisting of Cl or alkyl, alkoxyalkyl, substituted alkyl, cycloalkyl;
R⁶ and R⁹ are selected from H or F with the proviso that at least one of R⁶ and R⁹ are hydrogen;
R¹⁰, R¹¹ are both methyl, or linked to form a cyclopropyl, cyclobutyl or cyclopentyl group;
R¹² is (CH₂)ₘ—R¹³;
m is selected from 0, 1 or 2;

R¹³ is selected from hydrogen, hydroxyl, lower alkyl, lower alkoxy, lower cycloalkenyl, substituted cycloalkenyl, lower cycloalkyl, substituted alkylhydroxyalkylamino, substituted cycloalkyl, aryl, substituted aryl, hetereoaryl, substituted heteroaryl, hetereocycle or substituted heterocycle;

R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;

R⁴ is selected from the group consisting of H, lower alkyl, substituted lower alkyl and lower cycloalkyl,
and the enantiomers thereof or a pharmaceutically acceptable salt or ester thereof.

Compounds prepared according to the invention include:
rac(2S,3R,4R,5R)-4-(3-chloro-phenyl)-3-(4-chloro-phenyl)-2-(2,2-dimethyl-propyl)-5-hydroxymethyl-pyrrolidine-3-carbonitrile,
rac(2S,3R,4R,5R)-4-(3-chloro-phenyl)-3-(4-chloro-phenyl)-2-(2,2-dimethyl-propyl)-5-hydroxymethyl-1-((3R,4S,5R)-3,4,5,6-tetrahydroxy-hexyl)-pyrrolidine-3-carbonitrile,
rac(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile,
rac(2S,3R,4S,5R)-4-(3-chloro-2-fluoro-phenyl)-3-(4-chloro-2-fluoro-phenyl)-2-(2,2-dimethyl-propyl)-5-hydroxymethyl-1-((3R,4S,5R)-3,4,5,6-tetrahydroxy-hexyl)-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid ethyl ester,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile,
epimers 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester,
chiral(2S,3R,4S,5R)-4-(3-chloro-2-fluoro-phenyl)-3-(4-chloro-2-fluoro-phenyl)-2-(2,2-dimethyl-propyl)-1-[4-(2-hydroxy-ethoxy)-benzyl]-5-hydroxymethyl-pyrrolidine-3-carbonitrile,
chiral(1R,2R)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid,
chiral(1S,2S)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(3-trimethylsilanyl-prop-2-ynyl)-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzamide trifluoroacetate salt,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(5-methyl-furan-2-yl)-propyl]-pyrrolidine-3-carbonitrile trifluoroacetate salt,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid methyl ester,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(tetrahydro-pyran-4-yl)-propyl]-pyrrolidine-3-carbonitrile,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(1-trityl-1H-imidazol-4-yl)-propyl]-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-3-methoxy-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-benzoic acid,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[2-(tetrahydro-pyran-4-yl)-ethyl]-pyrrolidine-3-carbonitrile,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[4-(6-methyl-4,8-dioxo-[1,3,6,2]dioxazaborocan-2-yl)-benzyl]-pyrrolidine-3-carbonitrile and
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(4-methyl-4-nitro-pentyl)-pyrrolidine-3-carbonitrile.

Compounds disclosed herein and covered by formula I, above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

### Dosages

The compounds of the present invention are inhibitors of MDM2-p53 interactions and are thus useful in the treatment or control of cell proliferative disorders, in particular chemoprevention of cancer. Chemoprevention is defined as inhibiting the development of invasive cancer by either blocking the initiating mutagenic event or by blocking the progression of pre-malignant cells that have already suffered an insult of inhibiting tumor relapse. These compounds and formulations containing said compounds are anticipated to be particularly useful in the treatment or control of solid tumors, such as, for example, breast, colon, lung and prostate tumors.

A “therapeutically effective amount” or “effective amount” of a compound in accordance with this invention means an amount of compound that is effective to prevent, alleviate or ameliorate symptoms of disease or prolong the survival of the subject being treated.

The therapeutically effective amount or dosage of a compound according to this invention can vary within wide limits. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

### Compositions/Formulations

In an alternative embodiment, the present invention includes pharmaceutical compositions comprising at least one compound of formula I, or a pharmaceutically acceptable salt thereof and a pharmaceutically acceptable excipient and/or carrier.

These pharmaceutical compositions can be suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

The pharmaceutical preparations of the invention can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain other therapeutically valuable substances, including additional active ingredients other than those of formula I.

General Synthesis of N-substituted 5-hydroxypyrrolidines

The present invention provides methods for the synthesis of the substituted N-substituted 5-hydroxypyrrolidines of the invention.

The compounds of the invention can be prepared by processes known in the art. Suitable processes for synthesizing these compounds are also provided in the examples. Generally, compounds of formula I can be synthesized according to one of the below described synthetic routes.

The key transformations are a convergent [2+3] cycloaddition of imine A and activated olefin B to generate pyrrolidine-3-carbonitrile compounds C in a stereoselective manner. Compound C then can be used directly to make alcohol D or resolved first and then used to make chiral alcohol D. Compound D was then reacted with aldehyde or a suitable alkylation reagent to generate the desired target I.

The starting materials are either commercially available or can be synthesized by methods known to those of ordinary skill in the art. Preparations of intermediates A and B are illustrated in Schemes 1 and 2 below. In general an appropriately selected aldehyde or ketone can be reacted with glycine tert-butyl ester or glycine methyl ester to generate imine A as a crude product (see Scheme 1 below).

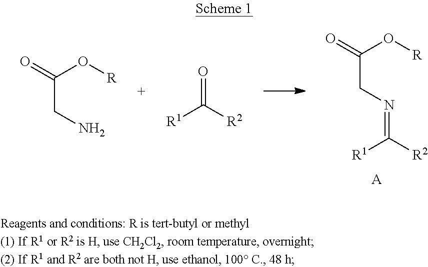

An intermediate of formula B can be made from a base-catalyzed condensation reaction of appropriately selected substituted-phenyl acetonitriles and aldehydes. The reaction proceeds in a highly stereoselective manner with the Z-isomer as the major or exclusive product (see scheme 2 below).

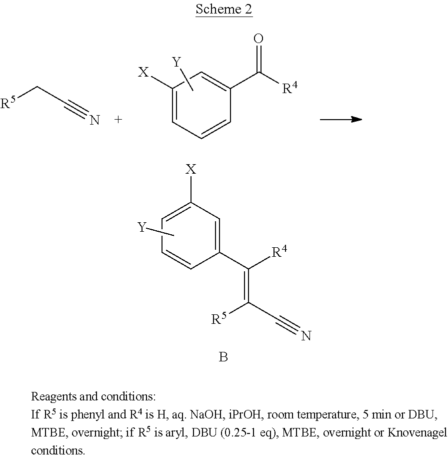

As is illustrated in Scheme 3 below, pyrrolidines of formula C can be made from intermediates A and B by a convergent 1,3-dipolar cycloaddition reaction mediated by lewis acid AgF and triethylamine, followed by hydrolysis. The [2+3] cycloaddition reactions of azomethine ylides 1,3-dipoles (that were generated from reacting intermediate A with AgF) with olefinic dipolarophiles for formula C to form pyrrolidine ring formation are described in the literature, including Jorgensen, K. A. et al (Org. Lett. 2005, Vol 7, No. 21, 4569-4572), Grigg, R. et al (Tetrahedron, 1992, Vol 48, No. 47, 10431-10442; Tetrahedron, 2002, Vol 58, 1719-1737), Schreiber, S. L. et al (J. Am. Chem. Soc., 2003, 125, 10174-10175), and Carretero, J. C. et al (Tetrahedron, 2007, 63, 6587-6602). Compounds of formula C are subsequently converted to compounds of formula D by reduction of the ester of C with various reducing agents using NaBH₄ or LiBH₄. Compound D can be further reacted by reductive amination conditions in the presence of an aldehyde to give E (see Scheme 3 below).

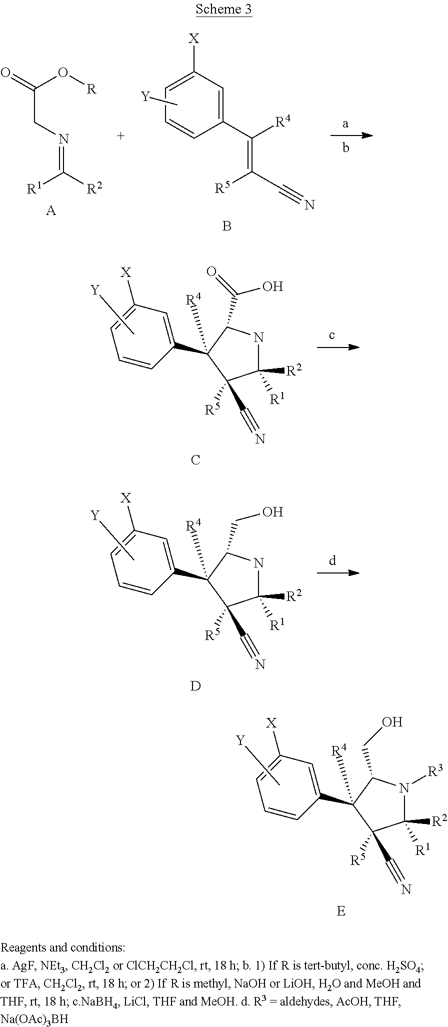

Racemic C can be readily resolved into two optically pure or enriched chiral enantiomers C1 and C2 by separation using chiral Super Fluid Chromatography (SFC). (see Scheme 4 below).

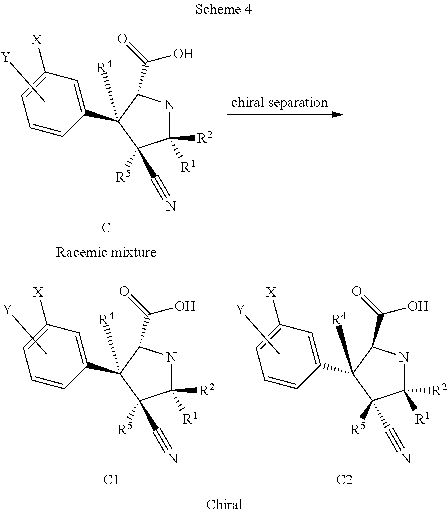

Compound E1 can be further reacted by Songashira conditions to give F (see Scheme 5 below).

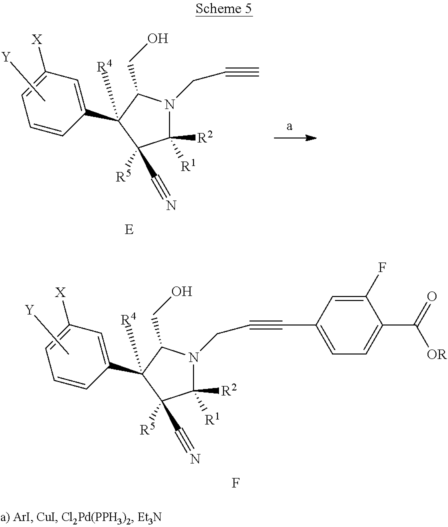

Resolution methods are well known, and are summarized in “Enantiomers, Racemates, and Resolutions” (Jacques, J. et. al. John Wiley and Sons, NY, 1981). Methods for chiral HPLC are also well known, and are summarized in “Separation of Enantiomers by Liquid Chromatographic Methods” (Rirkle, W. H. and Finn, J in Asymmetric Synthesis' Vol. 1, Morrison, J. D., Ed. Academic Press, In., NY 1983, pp. 87-124).

Converting a Compound of Formula I that Bears a Basic Nitrogen into a Pharmaceutically Acceptable Acid Addition Salt

The optional conversion of a compound of formula I that bears a basic nitrogen into a pharmaceutically acceptable acid addition salt can be effected by conventional means. For example, the compound can be treated with an inorganic acid such as for example hydrochloric acid, hydrobromic acid, sulfuric acid, nitric acid, phosphoric acid, or with an appropriate organic acid such as acetic acid, citric acid, tartaric acid, methanesulfonic acid, p-toluene sulfonic acid, or the like.

Converting a Compound of Formula I that Bears a Carboxylic Acid Group into a Pharmaceutically Acceptable Alkali Metal Salt

The optional conversion of a compound of formula I that bears a carboxylic acid group into a pharmaceutically acceptable metal salt can be effected by conventional means. For example, the compound can be treated with an inorganic base such as lithium hydroxide, sodium hydroxide, potassium hydroxide, or the like.

### Crystal Forms

When the compounds of the invention are solids, it is understood by those skilled in the art that these compounds, and their salts, may exist in different crystal or polymorphic forms, all of which are intended to be within the scope of the present invention and specified formulas.

### EXAMPLES

The compounds of the present invention may be synthesized according to known techniques. The following examples and references are provided to aid the understanding of the present invention. The examples are not intended, however, to limit the invention, the true scope of which is set forth in the appended claims. The names of the final products in the examples were generated using Isis AutoNom 2000.

### Abbreviations Used in the Examples

### HRMS: High Resolution Mass Spectrometry

### LCMS: Liquid Chromatography Mass Spectrometry

HATU: 2-(7-Azabenzotriazol-1-yl)-n,n,n′,n′-tetramethyluronium hexafluorophosphate
RT (or rt) Room temperature
DBU: 1,8-Diazabicyclo[5,4,0]undec-7-ene
DIBAL: Diisobutylalumiunum hydride
iPA: Isopropyl alcohol
ASDI: ASDI-Intermediates (company name)
RP-HPLC: Reverse phase HPLC

### Min: Minutes

### H or hrs: Hours

### GST; Glutathione S-transferase

TRF: Time resolved fluorescence

### Example 1

### Preparation of rac(2S,3R,4R,5R)-4-(3-chloro-phenyl)-3-(4-chloro-phenyl)-2-(2,2-dimethyl-propyl)-5-hydroxymethyl-pyrrolidine-3-carbonitrile

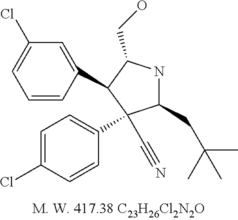

In a round-bottomed flask, rac(2R,3R,4R,5S)-3-(3-chloro-phenyl)-4-(4-chloro-phenyl)-4-cyano-5-(2,2-dimethyl-propyl)-pyrrolidine-2-carboxylic acid ethyl ester (221 mg, 0.48 mmol) was dissolved in THF (20 mL) and ethanol (20 mL) then lithium chloride (0.56 g, 13.21 mmol) and sodium borohydride (0.4 g, 1.5 mmol) were added and stirred at room temperature overnight. The reaction mixture was diluted with 0.1 N NaOH and extracted with EtOAc and washed with water. The organic layer was separated and concentrated under reduced pressure to afford crude product that was purified by RP-HPLC (20-95% acetonitrile/water) to afford rac(2S,3R,4R,5R)-4-(3-chloro-phenyl)-3-(4-chloro-phenyl)-2-(2,2-dimethyl-propyl)-5-hydroxymethyl-pyrrolidine-3-carbonitrile (90 mg, 45%) as an off-white powder.

HRMS (ES⁺) m/z Calcd for C₂₃H₂₆Cl₂N₂O+H[(M+H)]: calc: 417.1495. found: 417.1493.

### Example 2

### Preparation of rac(2S,3R,4R,5R)-4-(3-chloro-phenyl)-3-(4-chloro-phenyl)-2-(2,2-dimethyl-propyl)-5-hydroxymethyl-1-((3R,4S,5R)-3,4,5,6-tetrahydroxy-hexyl)-pyrrolidine-3-carbonitrile

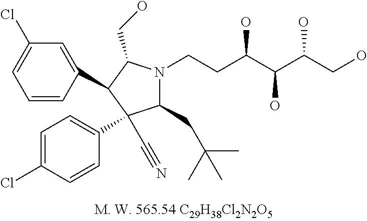

In a round-bottomed flask rac(2S,3R,4R,5R)-4-(3-chloro-phenyl)-3-(4-chloro-phenyl)-2-(2,2-dimethyl-propyl)-5-hydroxymethyl-pyrrolidine-3-carbonitrile (20 mg, 0.048 mmol), 2-deoxy-D-glucose (26 mg, 0.158 mmol), sodium triacetoxyborohydride (Fluka, 120 mg, 0.56 mmol) were combined with dichloromethane/THF (1:1, 2 mL) and stirred at rt. The reaction was concentrated with cosolvent toluene (5 mL, to azeotrope water out of reaction flask) under reduced pressure to afford an colorless oil that was purified by RP-HPLC (10-80% acetonitrile/water) to afford rac(2S,3R,4R,5R)-4-(3-chloro-phenyl)-3-(4-chloro-phenyl)-2-(2,2-dimethyl-propyl)-5-hydroxymethyl-1-((3R,4S,5R)-3,4,5,6-tetrahydroxy-hexyl)-pyrrolidine-3-carbonitrile as an off-white powder (7.9 mg, 29%). HRMS (ES⁺) m/z Calcd for C₂₉H₃₈Cl₂N₂O₅+H[(M+)¹⁺]: calc: 565.2231. found: 565.2227.

### Example 3

### Preparation of rac(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile

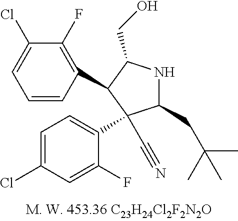

In a round-bottomed flask, rac(2R,3R,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester (230 mg, 478 μmol) was dissolved in THF (1 mL) and methanol (5 mL) then sodium borohydride (0.4 g, 10.6 mmol) were added and stirred at room temperature overnight. The reaction was incomplete, added LiCl (50 mg, 1.19 mmol) and methanol 2 mL then an additional NaBH₄ (0.4 g, 10.6 mmol) portionwise over 1 h. The reaction mixture was diluted with 0.1 N NaOH and extracted with EtOAc and washed with water. The organic layer was separated and concentrated under reduced pressure to afford rac(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (214 mg, 98.8%) as an white solid.

HRMS (ES⁺) m/z Calcd for C₂₃H₂₄Cl₂F₂N₂O+H[(M+H)]: calc: 453.1307. found: 453.1304.

### Example 4

### Preparation of rac(2S,3R,4S,5R)-4-(3-chloro-2-fluoro-phenyl)-3-(4-chloro-2-fluoro-phenyl)-2-(2,2-dimethyl-propyl)-5-hydroxymethyl-1-((3R,4S,5R)-3,4,5,6-tetrahydroxy-hexyl)-pyrrolidine-3-carbonitrile

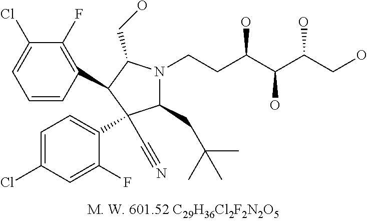

In a round-bottomed flask rac(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (142 mg, 313 μmol), 2-deoxy-D-glucose (286 mg, 1.74 mmol), and acetic acid (0.1 mL) were combined with dichloromethane/THF (1:1, 2.86 mL) and stirred at rt. Then sodium triacetoxyborohydride (Fluka, 400 mg, 313 μmol) was added and stirred for 72 h. Multiple pdts by LCMS. Concentrated under reduced pressure to afford a crude solid that was purified by RP-HPLC (20-95% acetonitrile/water, used lyophylyzer to concentrate pure samples) to afford rac(2S,3R,4S,5R)-4-(3-chloro-2-fluoro-phenyl)-3-(4-chloro-2-fluoro-phenyl)-2-(2,2-dimethyl-propyl)-5-hydroxymethyl-1-((3R,4S,5R)-3,4,5,6-tetrahydroxy-hexyl)-pyrrolidine-3-carbonitrile as an white powder (10.2 mg, 5.4%). HRMS (ES⁺) m/z Calcd for C₂₉H₃₆Cl₂F₂N₂O₅+H[(M+)¹⁺]: calc: 601.2042. found: 601.2043.

### Example 5

### Preparation of chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid

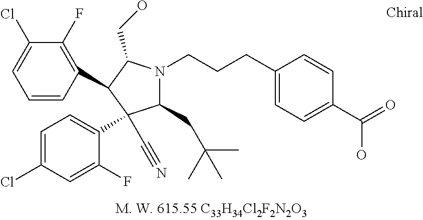

In a round-bottomed flask chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid ethyl ester (90 mg, 140 μmol), 2N KOH (1 mL, 2 mmol) were dissolved in ethanol (10 mL) and stirred overnight at rt. The reaction was concentrated under reduced pressure to afford a crude solid that was purified by RP-HPLC (30-95% acetonitrile/water, used lyophylyzer to concentrate pure samples) to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid as white powder (42 mg, 48.8%). HRMS (ES⁺) m/z Calcd for C₃₃H₃₄Cl₂F₂N₂O₃+H[(M+)¹⁺]: calc: 615.1988. found: 615.1986.

### Example 6

### Preparation of chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid ethyl ester

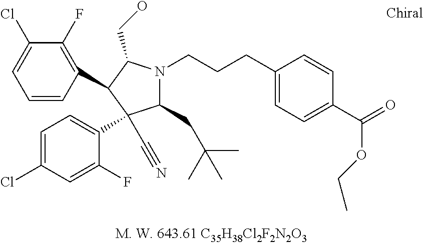

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (0.115 g, 254 μmol), 3-(4-carboethoxy)phenylpropanal (0.4 g, 1.94 mmol), and acetic acid (0.6 mL, 254 μmol) were combined with dichloromethane/THF (1:1, 4 mL) and stirred at rt. Then sodium triacetoxyborohydride (Fluka, 400 mg, 1.89 mmol) was added and stirred for 72 h at rt. Concentrated under reduced pressure to afford a crude solid that was purified by column chromatography (8 g, Analogix, 1-30% ethyl acetate/heptane) to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid ethyl ester as an white powder (150 mg, 91.9%). HRMS (ES⁺) m/z Calcd for C₃₅H₃₈Cl₂F₂N₂O₃+H[(M+)¹⁺]: calc: 643.2301. found: 643.2299.

### Example 7

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile

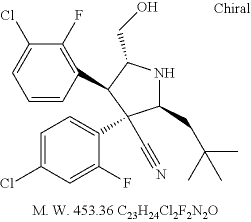

In a round-bottomed flask, chiral(2R,3R,4R,5S)-3-(3-chloro-2-fluoro-phenyl)-4-(4-chloro-2-fluoro-phenyl)-4-cyano-5-(2,2-dimethyl-propyl)-pyrrolidine-2-carboxylic acid methyl ester (230 mg, 478 μmol) was dissolved in THF (8 mL) and methanol (30 mL) then LiCl (240 mg, 5.67 mmol) was added, followed by sodium borohydride (895 g, 23.7 mmol stirring at room temperature overnight. Monitor by LCMS (10-100% acetonitrile/water) until complete (14 h). The reaction mixture was diluted with 0.1 N NaOH and extracted with EtOAc. The organic layer was separated, dried with Na2SO4, filtered and concentrated under reduced pressure to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (0.85 g, 99.2%) as an white solid. LCMS MS (ES⁺) m/z Calcd for C₂₃H₂₄Cl₂F₂N₂O+H[(M+H)]: calc: 453.1307. found: 453.1308.

### Example 8

### Preparation of epimers 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester

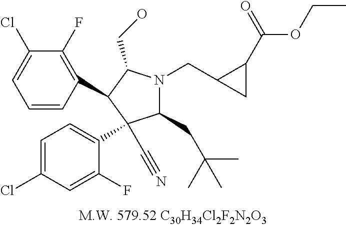

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (50 mg, 110 μmol), ethyl 2-formyl-1-cyclopropanecarboxylate (predominately trans, 0.15 g, 1.06 mmol), and acetic acid (1.5 mL) were combined and stirred at rt. Then sodium triacetoxyborohydride (Fluka, 140 mg, 661 μmol) was added and stirred for 72 h at rt. Concentrated under reduced pressure to afford a crude solid that was purified by RP-HPLC (40-95% acetonitrile/water) to epimers 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester as an white powder (42 mg, 65.7%). HRMS (ES⁺) m/z Calcd for C₃₀H₃₄Cl₂F₂N₂O₃H [(M+¹⁺]: calc: 579.1988. found: 579.1986.

### Example 9

### Preparation of chiral(2S,3R,4S,5R)-4-(3-chloro-2-fluoro-phenyl)-3-(4-chloro-2-fluoro-phenyl)-2-(2,2-dimethyl-propyl)-1-[4-(2-hydroxy-ethoxy)-benzyl]-5-hydroxymethyl-pyrrolidine-3-carbonitrile

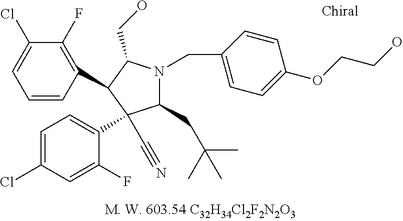

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (50 mg, 110 μmol), 4-(2-hydroxyethoxy)benzaldehyde (0.158 g, 0.951 mmol), and acetic acid (1.5 mL) were combined and stirred at rt. Then sodium triacetoxyborohydride (Fluka, 160 mg, 755 μmol) was added and stirred for 14 h at rt. The reaction was diluted with ethyl acetate and 0.1N NaOH, separated, the organic layer was washed with water (3×), the organic layer was concentrated under reduced pressure to afford 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester as an white powder (12 mg, 18%). HRMS (ES⁺) m/z Calcd for C₃₂H₃₄Cl₂F₂N₂O₃H[(M+)¹⁺]: calc: 603.1988. found: 603.1988.

### Example 10 a & b

### Preparation of chiral(1R,2R)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid & chiral(1S,2S)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid

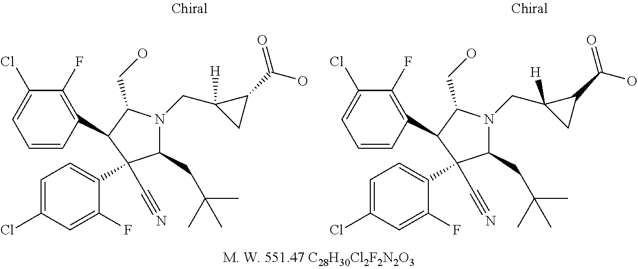

In a round-bottomed flask epimers 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester (36.4 mg, 62.8 μmol), 2N LiOH (3 mL, 1.5 mmol) were dissolved in THF (3 mL) and stirred 4 h at rt. The reaction was diluted with ethyl acetate (6 mL) and water (1 mL). The organic layer was separated and concentrated under reduced pressure to afford a crude solid that was purified by RP-HPLC (40-95% acetonitrile/water, used lyophylyzer to concentrate pure samples) to afford two products chiral (1R,2R)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid (5 mg, 12%) as an off-white solid

HRMS (ES⁺) m/z Calcd for C₂₈H₃₀Cl₂F₂N₂O₃+H[(M+)¹⁺]: calc: 551.1675. found: 551.1674. and chiral(1S,2S)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid as an off-white solid (3.1 mg, 7.42%). HRMS (ES⁺) m/z Calcd for C₂₈H₃₀Cl₂F₂N₂O₃+H[(M+)¹⁺]: calc: 551.1675. found: 551.1674.

### Example 11

### Preparation of chiral(2S,3R,4S,5R)-4-(3-Chloro-2-fluoro-phenyl)-3-(4-chloro-2-fluoro-phenyl)-2-(2,2-dimethyl-propyl)-5-hydroxymethyl-1-(3-trimethylsilanyl-prop-2-ynyl)-pyrrolidine-3-carbonitrile

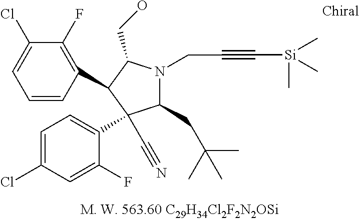

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (30 mg, 66.2 μmol), 3-trimethylsilylpropynal (33.4 mg, 0.265 mmol), and acetic acid (1.5 mL) were combined and stirred at rt. Then sodium triacetoxyborohydride (Fluka, 120 mg, 566 μmol) was added and stirred for 14 h at rt. The reaction was diluted with ethyl acetate and 0.1N NaOH, separated, the organic layer was washed with water (3×), the organic layer was concentrated under reduced pressure to afford crude mixture that was purified by RP-HPLC (40-95% acetonitrile/water) to afford chiral(2S,3R,4S,5R)-4-(3-Chloro-2-fluoro-phenyl)-3-(4-chloro-2-fluoro-phenyl)-2-(2,2-dimethyl-propyl)-5-hydroxymethyl-1-(3-trimethylsilanyl-prop-2-ynyl)-pyrrolidine-3-carbonitrile as an white powder (21.5 mg, 57.6%). HRMS (ES⁺) m/z Calcd for C₂₉H₃₄Cl₂F₂N₂OSi H[(M+)¹⁺]: calc: 563.1859. found: 563.1858.

### Example 12

### Preparation of chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzamide trifluoroacetate salt

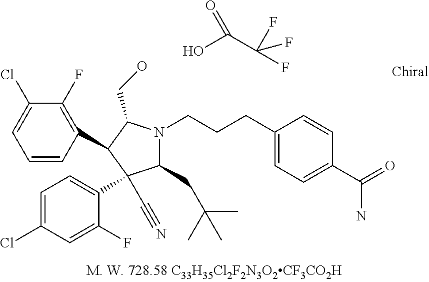

In a round-bottomed flask chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid (60 mg, 97.5 μmol), ammonia ([0.5M], 1.97 mL, 975 μmol) and HATU [2-(1H-7-Azabenzotriazol-1-yl)-1,1,3,3-tetramethyl uronium hexafluorophosphate Methanaminium, 51.9 mg, 136 μmol] was dissolved in dichloromethane (5 mL) and stirred overnight at rt. The reaction was concentrated under reduced pressure to afford a crude solid that was purified by RP-HPLC (30-95% acetonitrile/water, used lyophylyzer to concentrate pure samples) to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzamide trifluoroacetate salt as white powder (23 mg, 32.4%). HRMS (ES⁺) m/z Calcd for C₃₃H₃₄Cl₂F₂N₂O₃+H[(M+)¹⁺]: calc: 615.1988. found: 615.1983.

### Example 13

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(5-methyl-furan-2-yl)-propyl]-pyrrolidine-3-carbonitrile trifluoroacetate salt

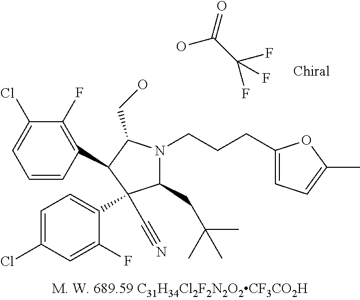

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (50 mg, 110 μmol), 3-(5-methylfuran-2-yl)propanal (81 mg, 0.441 mmol), and acetic acid (4 mL) were combined and stirred at rt. Then sodium triacetoxyborohydride (234 mg, 1.1 mmol) was added and stirred for 14 h at rt. The reaction was diluted with ethyl acetate and 0.1N NaOH, separated, the organic layer was concentrated under reduced pressure to afford crude mixture that was purified by RP-HPLC (30-95% acetonitrile/water) to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(5-methyl-furan-2-yl)-propyl]-pyrrolidine-3-carbonitrile trifluoroacetate salt as a light brown powder (12 mg, 15.8%). HRMS (ES⁺) m/z Calcd for C₃₁H₃₄Cl₂F₂N₂O₂H[(M+)¹⁺]: calc: 575.2038. found: 575.2036.

### Example 14

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3

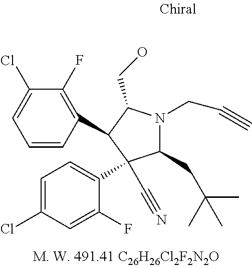

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(3-trimethylsilanyl-prop-2-ynyl)-pyrrolidine-3-carbonitrile (123 mg, 218 mmol), 2N LiOH (1 mL, 2 mmol) were dissolved in THF (3 mL) and Methanol (1 mL) and stirred 5 h at rt. The reaction was diluted with ethyl acetate and water. The organic layer was separated and concentrated under reduced pressure to afford chiral (2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (87.8 mg, 81.8%) as an off-white foam.

HRMS (ES⁺) m/z Calcd for C₂₆H₂₆Cl₂F₂N₂O+H[(m+)¹⁺]: calc: 491.1463. found: 491.1463.

### Example 15

Preparation chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid methyl ester

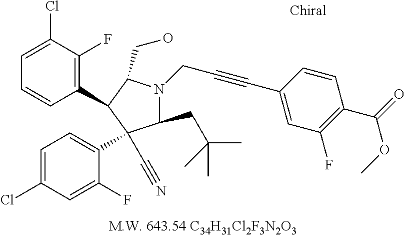

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (30 mg, 61.1 μmol), methyl 2-fluoro-4-iodobenzoate (17.1 mg, 61.1 μmol, Eq: 1.00) were dissolved in toluene (1.2 ml) then cuprous iodide (2.33 mg, 12.2 μmol) dichlorobis(triphenylphosphine) palladium (II) (1.29 mg, 1.83 μmol) and triethylamine (6.16 mg, 8.51 μmol, 61.1 μmol) were added and stirred under nitrogen atmosphere at 25° C. for 16 hours (protect from light with aluminum foil around flask during reaction conditions). Work up by filtration through celite, wash with EtOAc, mixture concentrated under reduced vacuum to yield a crude oil. Purification by RP-HPLC (40-95% acetonitrile/water) to afford chiral methyl 4-(3-((2S,3R,4S,5R)-4-(3-chloro-2-fluorophenyl)-3-(4-chloro-2-fluorophenyl)-3-cyano-5-(hydroxymethyl)-2-neopentylpyrrolidin-1-yl)prop-1-ynyl)-2-fluorobenzoate (17.3 mg, 26.9 μmol, 44.0% yield) as an off-white foam. HRMS (ES⁺) m/z Calc for C₂₆H₂₆Cl₂F₂N₂O+H[(M+)¹⁺]: calc: 643.1737. found: 643.1735.

### Example 16

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(tetrahydro-pyran-4-yl)-propyl]-pyrrolidine-3-carbonitrile

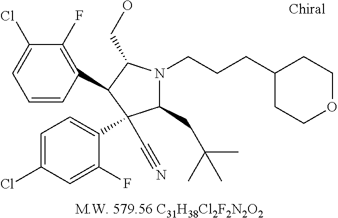

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (100 mg, 221 μmol), 3-(tetrahydro-2H-pyran-4-yl) propanal (94.1 mg, 0.862 mmol), and acetic acid (5 mL) were combined and stirred at rt. Then sodium triacetoxyborohydride (103 mg, 0.485 mmol) was added and stirred for 14 h at rt. The reaction was diluted with ethyl acetate and 0.1N NaOH, separated, the organic layer was concentrated under reduced pressure to afford crude mixture. Purify by RP-HPLC (25-95% acetonitrile/water) to afford trifluoroacetate salt of product, that was free-based with ethylacetate and NaHCO₃(s) solution, organic layer separated and concentrated under reduced pressure to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(tetrahydro-pyran-4-yl)-propyl]-pyrrolidine-3-carbonitrile as na off-white solid (31 mg, 24.2%). HRMS (ES⁺) m/z Calcd for C₃₁H₃₈Cl₂F₂N₂O₂H[(M+)¹⁺]: LCMS (7 min-C18-PosNeg50-100-Grad-LC RT=5.42, [(M+)¹⁺]: 579.1, 581.0)

### Example 17

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(1-trityl-1H-imidazol-4-yl)-propyl]-pyrrolidine-3-carbonitrile

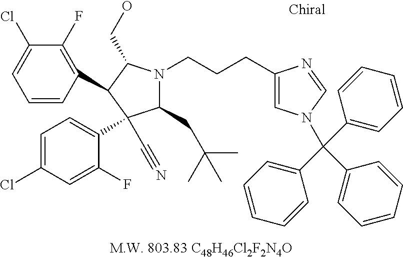

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (50 mg, 110 μmol), 3-(1-trityl-1H-imidazol-4-yl)propanal (150 mg, 0.409 mmol), and acetic acid (4.2 mL) were combined and stirred at rt. Then sodium triacetoxyborohydride (234 mg, 1.1 mmol) was added and stirred for 14 h at rt. The reaction was diluted with ethyl acetate and 0.1N NaOH, the organic layer was separated and washed with water then concentrated under reduced pressure to afford crude mixture. Purification by RP-HPLC (30-95% acetonitrile/water) to afford chiral (2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(1-trityl-1H-imidazol-4-yl)-propyl]-pyrrolidine-3-carbonitrile as a colorless foam (7 mg, 7.9%). HRMS (ES⁺) m/z Calcd for C₄₈H₄₆Cl₂F₂N₄O H[(M+)¹⁺]: ESMS ([(M+)¹⁺]: 803.3, 563.1, 561.2, 322.3, 302.7, 301.8.

### Example 18

### Preparation of chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid

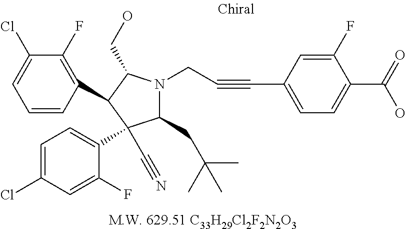

In a 15 mL round-bottomed flask, methyl 4-(3-((2S,3R,4S,5R)-4-(3-chloro-2-fluorophenyl)-3-(4-chloro-2-fluorophenyl)-3-cyano-5-(hydroxymethyl)-2-neopentylpyrrolidin-1-yl)prop-1-ynyl)-2-fluorobenzoate (36 mg, 55.9 μmol, Eq: 1.00) and 2N LiOH (1 mL, 2.00 mmol, Eq: 35.8) were combined with methanol (1 ml) and THF (3 mL) to give a colorless solution. The reaction was stirred for 3 h. Then diluted with Ethyl acetate and water. The organic layer was separated and concentrated under reduced pressure to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (12 mg, 34.1%) as an off-white solid. HRMS (ES⁺) m/z Calcd for C₃₃H₂₉Cl₂F₃N₂O₃+H[(M+)¹⁺]: calc: 629.1580. found: 629.1575.

### Example 19

Preparation chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoic acid

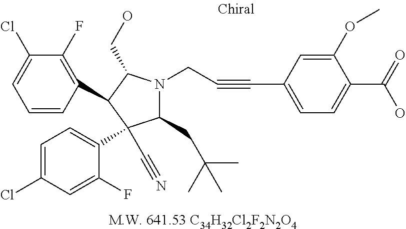

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (60 mg, 122 μmol), methyl 2-methoxy-4-iodobenzoate (35.7 mg, 122 μmol, Eq: 1.00) were dissolved in toluene (2.4 ml) then cuprous iodide (4.65 mg, 24.4 μmol) dichlorobis(triphenylphosphine) palladium (II) (2.57 mg, 3.66 μmol) and triethylamine (12.4 mg, 17 μL, 122 μmol) were added and stirred under nitrogen atmosphere at 25° C. for 16 hours (protect from light with aluminum foil around flask during reaction conditions). Work up by filtration through celite, wash with EtOAc, mixture concentrated under reduced vacuum to yield a crude oil. Purification by RP-HPLC (40-95% acetonitrile/water) to afford chiral methyl 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoate which was diluted with THF (4 mL) and methanol (2.4 mL) and reacted with 2N LiOH (2 mL) for 3 h at 25° C. with stirring. The reaction was then diluted with ethyl acetate and water, the organic layer was separated and concentrated under reduced pressure to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoic acid as an off-white solid (12 mg, 15.3%).

LCMS R_{T}=4.74 (MassLynx-7 min-C18-PosNeg50-100Grad-LC) m/z Calcd for C₃₄H₃₂Cl₂F₂N₂O₄+H[(M+)¹⁺]: 641.1

### Example 20

Preparation chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-3-methoxy-benzoic acid

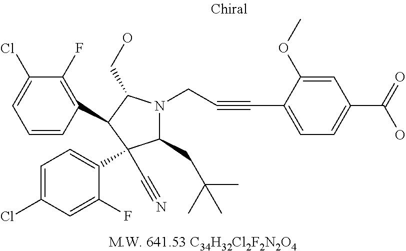

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (60 mg, 122 μmol), ethyl 3-methoxy-4-iodobenzoate (37.4 mg, 122 μmol, Eq: 1.00) were dissolved in toluene (2.4 ml) then cuprous iodide (4.65 mg, 24.4 μmol) dichlorobis(triphenylphosphine) palladium (II) (2.57 mg, 3.66 μmol) and triethylamine (12.4 mg, 17 μl, 122 μmol) were added and stirred under nitrogen atmosphere at 25° C. for 16 hours (protect from light with aluminum foil around flask during reaction conditions). Work up by filtration through celite, wash with EtOAc, mixture concentrated under reduced vacuum to yield a crude oil. Purification by RP-HPLC (40-95% acetonitrile/water) to afford chiral ethyl 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoate which was diluted with THF (4 mL) and methanol (2.4 mL) and reacted with 2N KOH (2 mL) for 2 h at 25° C. with stirring. The reaction was then diluted with ethyl acetate and water, the organic layer was separated and concentrated under reduced pressure to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-3-methoxy-benzoic acid as an off-white crystalline solid (6 mg, 7.67%).

LCMS R_{T}=4.89 (MassLynx-7 min-C18-PosNeg50-100Grad-LC) m/z Calcd for C₃₄H₃₂Cl₂F₂N₂O₄+H[(M+)¹⁺]: 641.1

### Example 21

Preparation chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-benzoic acid

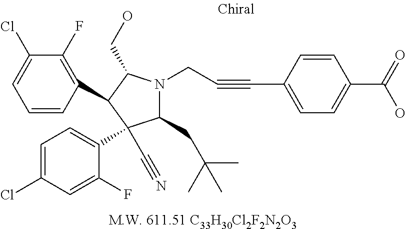

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile (60 mg, 122 μmol), methyl 4-iodobenzoate (32 mg, 122 μmol, Eq: 1.00) were dissolved in toluene (2.4 ml) then cuprous iodide (4.65 mg, 24.4 μmol) dichlorobis(triphenylphosphine) palladium (II) (2.57 mg, 3.66 μmol) and triethylamine (12.4 mg, 17 μl, 122 μmol) were added and stirred under nitrogen atmosphere at 25° C. for 16 hours (protect from light with aluminum foil around flask during reaction conditions). Work up by filtration through celite, wash with EtOAc, mixture concentrated under reduced vacuum to yield a crude oil. Purification by RP-HPLC (40-95% acetonitrile/water) to afford chiral ethyl 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoate which was diluted with THF (4 mL) and methanol (2.4 mL) and reacted with 2N LiOH (2 mL) for 3 h at 25° C. with stirring. The reaction was then diluted with ethyl acetate and water, the organic layer was separated and concentrated under reduced pressure to afford chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-benzoic acid as an off-white crystalline solid (6 mg, 8.04%).

LCMS R₁=4.93 (MassLynx-7 min-C18-PosNeg50-100Grad-LC) m/z Calcd for C₃₃H₃₀Cl₂F₂N₂O₃+H[(M+)¹⁺]: 611.0

### Example 22

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[2-(tetrahydro-pyran-4-yl)-ethyl]-pyrrolidine-3-carbonitrile

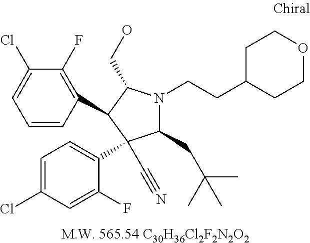

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (48 mg, 106 μmol), 2-(tetrahydro-2H-pyran-4-yl)acetaldehyde (100 mg, 0.78 mmol), and acetic acid (5 mL) were combined and stirred at rt. Then sodium triacetoxyborohydride (140 mg, 0.661 mmol) was added and stirred for 14 h at rt. The reaction was diluted with ethyl acetate and 0.1N NaOH, separated, the organic layer was concentrated under reduced pressure to afford crude mixture. Purify by RP-HPLC (25-95% acetonitrile/water) to afford trifluoroacetate salt of product, that was free-based with ethyl acetate and NaHCO₃(s) solution, organic layer separated and concentrated under reduced pressure to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[2-(tetrahydro-pyran-4-yl)-ethyl]-pyrrolidine-3-carbonitrile as an colorless crystalline solid (15.2 mg, 25.4%). HRMS MS (ES⁺) m/z Calc for C₃₀H₃₆Cl₂F₂N₂O₂H[(M+)¹⁺]: LCMS (7 min-C18-PosNeg50-100-Grad-LC RT=5.12, [(M+)¹⁺]: 565.1

### Example 23

### Preparation of chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[4-(6-methyl-4,8-dioxo-[1,3,6,2]dioxazaborocan-2-yl)-benzyl]-pyrrolidine-3-carbonitrile

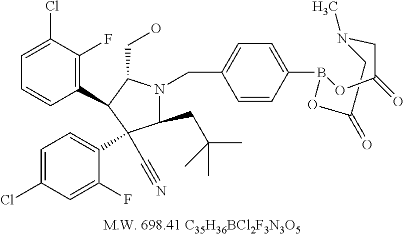

In a 25 mL round-bottomed flask, 4-Formyl Phenyl MIDA Boronic ester (44.9 mg, 172 μmol, Eq: 1.5), chiral(2S,3R,4S,5R)-4-(3-chloro-2-fluorophenyl)-3-(4-chloro-2-fluorophenyl)-5-(hydroxymethyl)-2-neopentylpyrrolidine-3-carbonitrile (52 mg, 115 μmol, Eq: 1.00), acetic acid (100 μL) and sodium triacetoxyborohydride (36.5 mg, 172 μmol, Eq: 1.5) were combined with DCE (5 ml) to give a light yellow solution. Reaction stirred 4 h at rt. Not complete, added more 4-Formyl Phenyl MIDA Boronic ester (60 mg, 230 μmol, 230 eq), triacetoxyborohydride (48.7 mg, 230 μmol, 2 eq) and acetic acid (1 mL) allowed to stir overnight. The reaction was diluted with ethyl acetate and 0.1N NaOH, organic separated and washed with water (3×). Organic layer was concentrated under reduced pressure and purified with column chromatography (8 g Analogix column, 0-100% Ethyl acetate/heptane) to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[4-(6-methyl-4,8-dioxo-[1,3,6,2]dioxazaborocan-2-yl)-benzyl]-pyrrolidine-3-carbonitrile (12 mg, 15%) as an off-white solid. HRMS (ES⁺) m/z Calcd for C₃₅H₃₆BCl₂F₂N₃O₅[(M+)¹⁺]: LCMS (7 min-C18-PosNeg50-100-Grad-LC RT=4.61, [(M+)¹⁺]: 698.1

### Example 24

### Preparation of chiral(2S,3R,4S,5R)-4-(3-chloro-2-fluoro-phenyl)-3-(4-chloro-2-fluoro-phenyl)-dimethyl-propyl)-5-hydroxymethyl-1-(4-methyl-4-nitro-pentyl)-pyrrolidine-3-carbonitrile

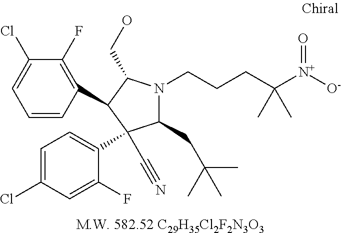

In a round-bottomed flask chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile (75 mg, 165 μmol), 4-methyl-4-nitropentanal (72 mg, 0.496 mmol), and acetic acid (1.56 mL) were combined and stirred at rt. Then sodium triacetoxyborohydride (105 mg, 0.496 mmol) was added and stirred for 14 h at rt. The reaction was diluted with ethyl acetate and 0.1N NaOH, separated and washed with water (3×), the organic layer was concentrated under reduced pressure to afford crude mixture. Purify by RP-HPLC (35-95% acetonitrile/water) to afford chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(4-methyl-4-nitro-pentyl)-pyrrolidine-3-carbonitrile as a white solid (79.4 mg, 82.4%). Calcd for C₂₉H₃₅Cl₂F₂N₃O₃[(M+)¹⁺]: LCMS (7 min-C18-PosNeg50-100-Grad-LC RT=5.69, [(M+)¹⁺]: 582.1

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53. Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing: 90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at 665 and 615 nm (Victor 5; Perkin Elmer Wallac). If not specified, the reagents were purchased from Sigma Chemical Co.

Activity data for some of the Example compounds expressed as IC₅₀: bsa: 0.02% are as follows:

| Example Number | IC₅₀: bsa: 0.02% |
|---|---|
| 1 | 2.86 |
| 2 | 0.74 |
| 3 | 0.971 |
| 4 | 0.164 |
| 5 | 0.212 |
| 6 | 4.13 |
| 7 | 0.182 |
| 8 | 2.32 |
| 9 | 2.11 |
| 10a | 0.087 |
| 10b | 0.242 |
| 11 | 1.08 |
| 12 | 1.52 |
| 13 | 1.67 |
| 14 | 2.36 |
| 15 | 0.887 |
| 16 | 0.489 |
| 17 | 0.215 |
| 18 | 0.0708 |

What is claimed:

1. A compound of formula I 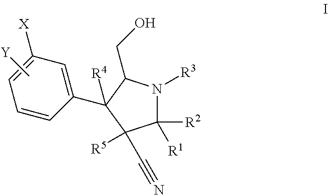
wherein
Y is selected from the group consisting of F, Cl, Br, I, cyano, nitro, lower alkyl, lower aklynyl and lower alkoxy;
X is H, F, Cl or CF₃;
R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
R⁵ are independently selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle
and the enantiomers or a pharmaceutically acceptable salt or ester thereof.

2. The compound of the claim 1 having the following stereochemistry 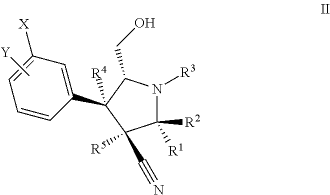
wherein
Y is selected from the group consisting of F, Cl, Br, I, cyano, nitro, lower alkyl, lower aklynyl and lower alkoxy;
X is H, F, Cl or CF₃;
R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
R⁵ are independently selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
and the enantiomers thereof or a pharmaceutically acceptable salt or ester thereof.

3. The compound of claim 2 having the formula 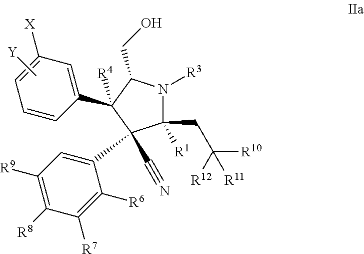
wherein,
Y is selected from the group consisting of F, Cl, Br, I, cyano, nitro, lower alkyl, lower aklynyl and lower alkoxy;
X is H, F, Cl or CF₃;
R⁸ is selected from the group consisting of F, Cl and Br;
R⁶, R⁷ and R⁹ are H or F with the proviso that at least two of R⁶, R⁷ and R⁹ are hydrogen;
R¹ and R² are independently selected from the group consisting of H, lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl and substituted cycloalkenyl;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
and the enantiomers thereof or a pharmaceutically acceptable salt or ester thereof.

4. The compound of claim 3 having the formula 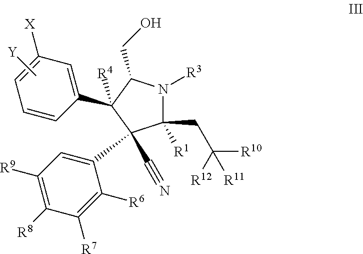
wherein,
R¹ is hydrogen;
Y is selected from the group consisting of F, Cl, Br, I, cyano, nitro, lower alkyl, lower aklynyl and lower alkoxy;
X is H, F, Cl or CF₃;
V is N or C
R⁸ is selected from the group consisting of Cl, alkyl, alkoxyalkyl, substituted alkyl and cycloalkyl;
R⁶ and R⁹ are selected from H or F with the proviso that at least one of R⁶ and R⁹ are hydrogen;
R¹⁰, R¹¹ are both methyl, or linked to form a cyclopropyl, cyclobutyl or cyclopentyl group;
R¹² is (CH₂)ₘ—R¹³;
m is selected from 0, 1 or 2;
R¹³ is selected from hydrogen, hydroxyl, lower alkyl, lower alkoxy, lower cycloalkenyl, substituted cycloalkenyl, lower cycloalkyl, substituted alkylhydroxyalkylamino, substituted cycloalkyl, aryl, substituted aryl, hetereoaryl, substituted heteroaryl, hetereocycle or substituted heterocycle;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
and enantiomers thereof or a pharmaceutically acceptable salt or ester thereof.

5. The compound of claim 4 having the formula 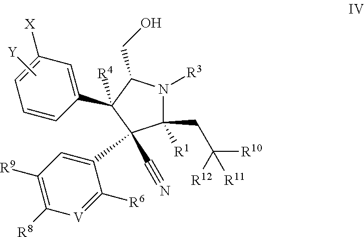
wherein,
Y is selected from the group consisting of F, Cl, Br, I, cyano, nitro, lower alkyl, lower aklynyl and lower alkoxy;
X is H, F, Cl or CF₃;
V is N
R⁸ is selected from the group consisting of Cl or alkyl, alkoxyalkyl, substituted alkyl and cycloalkyl;
R⁶ and R⁹ are selected from H or F with the proviso that at least one of R⁶ and R⁹ are hydrogen;
R¹⁰, R¹¹ are both methyl, or linked to form a cyclopropyl, cyclobutyl or cyclopentyl group;
R¹² is (CH₂)ₘ—R¹³;
m is selected from 0, 1 or 2;
R¹³ is selected from hydrogen, hydroxyl, lower alkyl, lower alkoxy, lower cycloalkenyl, substituted cycloalkenyl, lower cycloalkyl, substituted alkylhydroxyalkylamino, substituted cycloalkyl, aryl, substituted aryl, hetereoaryl, substituted heteroaryl, hetereocycle or substituted heterocycle;
R³ is selected from the group consisting of H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle and substituted heterocycle;
R⁴ is selected from the group consisting of H, lower alkyl, substituted lower alkyl and lower cycloalkyl,
and the enantiomers thereof or a pharmaceutically acceptable salt or ester thereof.

6. A compound of claim 1 selected from the group consisting of
rac(2S,3R,4R,5R)-4-(3-chloro-phenyl)-3-(4-chloro-phenyl)-2-(2,2-dimethyl-propyl)-5-hydroxymethyl-pyrrolidine-3-carbonitrile,
rac(2S,3R,4R,5R)-4-(3-chloro-phenyl)-3-(4-chloro-phenyl)-2-(2,2-dimethyl-propyl)-5-hydroxymethyl-1-((3R,4S,5R)-3,4,5,6-tetrahydroxy-hexyl)-pyrrolidine-3-carbonitrile,
rac(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile,
rac(2S,3R,4S,5R)-4-(3-chloro-2-fluoro-phenyl)-3-(4-chloro-2-fluoro-phenyl)-2-(2,2-dimethyl-propyl)-5-hydroxymethyl-1-((3R,4S,5R)-3,4,5,6-tetrahydroxy-hexyl)-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzoic acid ethyl ester,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-pyrrolidine-3-carbonitrile,
epimers 2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid ethyl ester,
chiral(2S,3R,4S,5R)-4-(3-chloro-2-fluoro-phenyl)-3-(4-chloro-2-fluoro-phenyl)-2-(2,2-dimethyl-propyl)-1-[4-(2-hydroxy-ethoxy)-benzyl]-5-hydroxymethyl-pyrrolidine-3-carbonitrile,
chiral(1R,2R)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid and
chiral(1S,2S)-2-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-ylmethyl]-cyclopropanecarboxylic acid.

7. A compound of claim 1 selected from the group consisting of
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(3-trimethylsilanyl-prop-2-ynyl)-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-propyl}-benzamide trifluoroacetate salt,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(5-methyl-furan-2-yl)-propyl]-pyrrolidine-3-carbonitrile trifluoroacetate salt,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-prop-2-ynyl-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid methyl ester,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(tetrahydro-pyran-4-yl)-propyl]-pyrrolidine-3-carbonitrile,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[3-(1-trityl-1H-imidazol-4-yl)-propyl]-pyrrolidine-3-carbonitrile,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-fluoro-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-2-methoxy-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-3-methoxy-benzoic acid,
chiral 4-{3-[(2S,3R,4S,5R)-4-(3-chloro-2-fluoro-phenyl)-3-(4-chloro-2-fluoro-phenyl)-3-cyano-2-(2,2-dimethyl-propyl)-5-hydroxymethyl-pyrrolidin-1-yl]-prop-1-ynyl}-benzoic acid,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[2-(tetrahydro-pyran-4-yl)-ethyl]-pyrrolidine-3-carbonitrile,
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-[4-(6-methyl-4,8-dioxo-[1,3,6,2]dioxazaborocan-2-yl)-benzyl]-pyrrolidine-3-carbonitrile and
chiral(2S,3R,4S,5R)-4-(3-chloro-2-fluoro-phenyl)-3-(4-chloro-2-fluoro-phenyl)-2-(2,2-dimethyl-propyl)-5-hydroxymethyl-1-(4-methyl-4-nitro-pentyl)-pyrrolidine-3-carbonitrile.

8. A pharmaceutical composition comprising a compound of claim 2, or a pharmaceutically acceptable salt or ester thereof, as an active ingredient together with a pharmaceutically acceptable carrier or excipient.
